(19) United States
(12) Patent Application Publication
(10) Pub. No.: US 2024/0376035 A1
(43) Pub. Date: Nov. 14, 2024

(54) METHOD FOR PRODUCING HESPERETIN DIHYDROCHALCONE

(71) Applicant: SYMRISE AG, Holzminden (DE)

(72) Inventors: Mona OLTMANNS, Holzminden (DE); Michael BACKES, Holzminden (DE); Birgit NOELTING, Bad Karlshafen (DE); Dietmar SCHATKOWSKI, Einbeck (DE); Stefan BRAND, Vahlbruch (DE); Carina DIERKS, Bad Driburg (DE); Artur DÜCK, Stadtoldendorf (DE)

(73) Assignee: SYMRISE AG, Holzminden (DE)

(21) Appl. No.: 18/778,622

(22) Filed: Jul. 19, 2024

Related U.S. Application Data
(63) Continuation-in-part of application No. PCT/EP2023/051399, filed on Jan. 20, 2023.
Application No. PCT/EP2023/051399 is a continuation of application No. PCT/EP2022/051305, filed on Jan. 21, 2022.

(51) Int. Cl. C07C 45/67; A23L 27/30; C07C 45/81

(52) U.S. Cl. CPC C07C 45/67; A23L 27/30; C07C 45/81

(57) ABSTRACT

The present invention relates to a method for producing hesperetin dihydrochalcone from hesperetin as starting material. Furthermore, the present invention relates to a method for imparting or modifying a sweet taste impression using the hesperetin dihydrochalcone formed by the methods described herein.

### CROSS-REFERENCE TO RELATED APPLICATIONS

This application claims priority to and is a continuation-in-part of PCT/EP2023/051399, filed Jan. 20, 2023, which claims the benefit of the filing date of PCT Application No. PCT/EP2022/051305, filed Jan. 21, 2022, the entirety of which is herein incorporated by reference.

### FIELD OF THE DISCLOSURE

The present invention relates to a method for producing hesperetin dihydrochalcone from hesperetin as starting material. Furthermore, the present invention relates to a method for imparting or modifying a sweet taste impression.

### BACKGROUND

Sweet tasting food and beverages with a high sugar content are very popular among consumers on a global scale. Common sugars used in such products are sucrose, glucose, lactose, fructose and mixtures thereof. Consuming high amounts of easily metabolizable carbohydrates leads to a rise in blood sugar and thus, if consumed in excess, lead to formation of fat deposits. Accordingly, this might lead to problems such as excess weight, various degrees of obesity, insulin resistance, late-onset diabetes and associated secondary diseases. Furthermore, consumption of the above-mentioned carbohydrates can also lead to severe dental health problems, as their bacterial degradation in anoxic regions in the oral cavity leads to the formation of fermentation products such as lactic acid. Consequently, there is a severe risk of damaging the enamel of adolescent and adult teeth.

Therefore, there is the constant incentive in the food and beverage industry to reduce the sugar content in a wide variety of different products in way that the consumers notice little to no difference. Retaining the originally sweet taste of a product, which is reduced in sugar content, can for instance be achieved by using different sweeteners.

Sweeteners are naturally occurring or synthetically produced sugar substitutes providing a pronounced sweet taste while containing significantly less food energy. Thus, there are zero-calorie (non-nutritive) and low-calorie (low-nutritive) sweeteners. Artificial sweeteners might be derived from plant extracts or produced by chemical synthesis. Some sweeteners are known as bulk sweeteners, such as sorbitol, mannitol or other sugar alcohols, and can also partially replace the properties of sugars. However, too frequent intake might lead to osmotically-induced digestion problems among some consumers. Especially high-intensity, non-nutritive sweeteners are very popular and suitable for retaining or imparting a sweet taste in sugar free products or products with reduced sugar content, as they can be used in comparatively low amounts. Despite that, even the use of such sweeteners at low concentrations might lead to taste-related off-notes not resembling the sweetness of sugar (e.g. sucralose, steviosode, cyclamate), a bitter and/or astringent aftertaste (e.g. acesulfame K, saccharin), or additional undesired flavour impressions (e.g. glycyrrhetinic acid ammonium salt).

On these grounds, it is a particular interest of the food and beverage industry to find and provide substances possessing an intensive sweet taste while none of the above-mentioned taste-related off notes are present.

In this context, dihydrochalcones as known sweetness modulating compounds are relevant, as they have been investigated as far back as 1979 with regard to their sweetening power in relation to their molecular structure (J. Chem. Senses 1979, 4(1), 35-47). It was elucidated that the 3-hydroxy-4-methoxy-phenylgroup is crucial for a powerful sweet taste while changing the substituents yields a loss of sweetening power.

Hesperetin dihydrochalcone (I) belongs to the group of dihydrochalcones and is a derivative of the flavanone-glycoside hesperetin (II). Hesperetin dihydrochalcone is a flavouring substance, which can be used in various applications to rebalance the profile of food products with a partially reduced sugar content or food products, which were artificially sweetened. Hesperetin dihydrochalcone possesses the crucial 3-hydroxy-4-methoxy-phenylgroup and also a 2,6-dihydroxy-substitution pattern, which is also assumed to be of importance for a strong sweetness impression (J. Med. Chem. 1981, 24(4), 408-428).

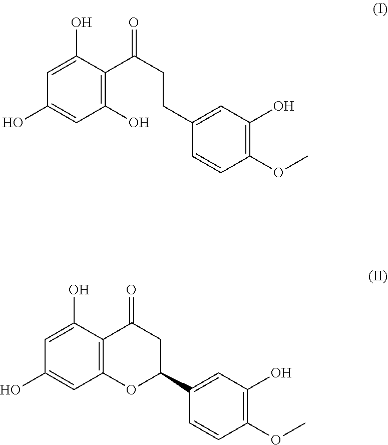 [SMILES: COc1ccc(CCC(=O)c2c(O)cc(O)cc2O)cc1O ; COc1ccc([C@@H]2CC(=O)c3c(O)cc(O)cc3O2)cc1O]

WO2007/107596 A1 describes different 4-hydroxydihydrochalcones and their salts for the intensification of sweet sensorial impression. For the sweetness-intensifying effects found for hesperetin dihydrochalcone, a 4-hydroxy substitution was shown to be necessary.

WO2017/186299 A1 discloses the use of hesperetin dihydrochalcone for suppressing a variety of different taste perceptions. Some application examples therein also disclose mixtures with various sweet-tasting substances, one of them being hesperetin. In these cases, hesperetin is always present in an amount much higher than hesperetin dihydrochalcone. In the general context of the application examples disclosed therein, it is immanent that sweet-tasting substances in general are present in an excess compared to hesperetin dihydrochalcone. Mixtures of hesperetin dihydrochalcone with corn syrup with increased fruit sugar content and other sweeteners are described in WO 2019/080990 A1. This application describes the use of an excess of sweeteners in comparison to hesperetin dihydrochalcone.

WO 2007/014879 A1 describes the use of hesperetin as an intensifier of the sweet flavour of sugar-reduced preparations. However, hesperetin shows a diminished effectivity in preparations having a high content of acid such as lemonades or fruit juices. However, these preparations are one of the main targets for sweetness improvement with low calorie sweeteners.

Presently, commercially relevant methods for the production of hesperetin dihydrochalcone use renewable citrus-based flavonoids as starting material, for example hesperidin or derivatives thereof. Therefore, crucial steps in these methods are (i) the ring-opening isomerization under basic conditions followed by (ii) hydrogenation employing large amounts of heavy metal catalysts with hydrogen as reducing agent. However, these methods require rather extreme reaction conditions, such as elevated temperatures and high pressure.

Alternatively, methods for converting hydroxyflavanones (e.g. hesperetin) into hydroxydihydrochalcones (e.g. hesperetin dihydrochalcone) including palladium catalysed transfer hydrogenation have rarely been described. Usually, these methods employ salts of formic acid in protic solvents as hydrogen source. Therefore, these methods generally do not require specialized equipment and do not necessitate the handling of gaseous hydrogen under high temperatures and elevated pressure.

Only a few methods using palladium catalysed transfer hydrogenation have been described. For instance, Ahmed and van Lier (J. Chem. Res. 2006, 584-585) described a method for the production of dihydrochalcones by hydrogenation of the respective chalcones employing ammonium formate as hydrogen source in combination with a palladium catalyst. However, this method uses the respective chalcones as starting material and thus requires the ring opening isomerization for the production of these chalcones to be carried out in a separate reaction step. Consequently, two reaction steps need to be performed, which increases the time and resources for obtaining the respective product.

Krishnamurty and Sathyanarayana (Synthetic Communication 1989, 19 (1&2), 119-123) described the conversion of hydroxy-and methoxyflavanones into the respective dihydrochalcones via catalytic transfer hydrogenation employing a palladium catalyst. This method includes both (i) the ring-opening isomerization as well as (ii) the subsequent hydrogenation step. However, the final yield of the produced dihydrochalcones when directly produced from hydroxy- and/or methoxyflavanones is comparatively low. Precisely, in the most efficient approach, a yield of 70 to 75% was achieved. Moreover, such methods have only been performed at a small scale and require comparatively inefficient reaction conditions. Thus, further improvement is required with regard to the applicability on industrial scale and with more efficient reaction conditions.

Accordingly, improved methods allowing the efficient production of dihydrochalcones, particularly of hesperetin dihydrochalcone, directly from hydroxyflavanones are strongly desired by the food industry.

Hence, it was the primary object of the present invention to provide a more efficient method for the production of hesperetin dihydrochalcone from hesperetin as starting material.

It was also an object of the present invention to provide a method for the production of hesperetin dihydrochalcone from hesperetin as starting material, which only involves comparatively mild reaction conditions.

It was another object of the present invention to provide a method for the production of hesperetin dihydrochalcone from hesperetin as starting material, which can be employed at a larger industrial scale.

### SUMMARY OF THE DISCLOSURE

It was surprisingly found that the primary object of the present invention could be solved with a method for producing hesperetin dihydrochalcone, comprising the following steps
i) providing hesperetin,
ii) providing a catalyst selected from the group consisting of palladium catalysts, ruthenium catalysts, gold catalysts, platinum catalysts, copper catalysts, cobalt catalysts and iron catalysts, preferably a palladium catalyst, preferably a Pd/C catalyst,
iii) providing a solvent, wherein the solvent comprises a solvent selected from the group consisting of methanol, ethanol, alkane diols with a total number of carbon atoms in the range of from 1 to 5, and mixtures thereof,
   preferably wherein the solvent comprises or consists of a mixture of water and one, two, three or more or all selected from methanol, ethanol, a 1,2-alkane diol with a total number of carbon atoms in the range of from 1 to 5, and a 1,3-alkane diol with a total number of carbon atoms in the range of from 1 to 5,
   preferably wherein the solvent is or comprises a mixture of water and ethanol,
iv) providing formic acid,
v) reacting the formic acid provided in step iv) to a formate, preferably wherein the formate is selected from the group consisting of potassium formate, calcium formate, magnesium formate, ammonium formate, sodium formate and mixtures thereof, especially preferably the formate is or comprises ammonium formate and/or sodium formate,
vi) mixing the components provided in steps i), ii) and iii) with the formate obtained in step v), to obtain a reaction mixture,
vii) heating the reaction mixture obtained in step vi) to a temperature in the range of from 30 to 60° C. to convert hesperetin to hesperetin dihydrochalcone,
viii) removing the catalyst, preferably the palladium catalyst, particularly preferably the Pd/C catalyst from the reaction mixture after step vii) is performed.

### DETAILED DESCRIPTION OF THE DISCLOSURE

The term “catalyst” as used herein refers to a chemical compound, which can be used in a method involving one or more chemical reactions in order to increase the reaction speed of a chemical reaction by decreasing the respective activation energy and which comprises an element selected from the group consisting of palladium, ruthenium, gold, platinum, copper, cobalt, and iron. Preferably, the element is supported on a support material selected from the group consisting of carbon, activated carbon, alumina, calcium carbonate, silica, silica-alumina.

The term “palladium catalyst” as used herein refers to a chemical compound comprising palladium, which can be used in a method involving one or more chemical reactions in order to increase the reaction speed of a chemical reaction by decreasing the respective activation energy. Preferably, the palladium is supported on a support material selected from the group consisting of carbon, activated carbon, alumina, calcium carbonate, silica, silica-alumina. The same applies accordingly with regard to further catalysts described herein.

Preferably, the catalyst is a palladium-carbon catalyst.

Preferably, the catalyst is a palladium-activated carbon catalyst.

Preferably, the catalyst is a palladium-alumina catalyst.

Preferably, the catalyst is a palladium-calcium carbonate catalyst.

Preferably, the catalyst is a palladium-silica catalyst.

Preferably, the catalyst is a palladium-silica-aluminum catalyst.

Preferably, the catalyst is a ruthenium-carbon catalyst.

Preferably, the catalyst is a ruthenium-activated carbon catalyst.

Preferably, the catalyst is a ruthenium-alumina catalyst.

Preferably, the catalyst is a ruthenium-calcium carbonate catalyst.

Preferably, the catalyst is a ruthenium-silica catalyst.

Preferably, the catalyst is a ruthenium-silica-aluminum catalyst.

Preferably, the catalyst is a gold-carbon catalyst.

Preferably, the catalyst is a gold-activated carbon catalyst.

Preferably, the catalyst is a gold-alumina catalyst.

Preferably, the catalyst is a gold-calcium carbonate catalyst.

Preferably, the catalyst is a gold-silica catalyst.

Preferably, the catalyst is a gold-silica-aluminum catalyst.

Preferably, the catalyst is a platinum-carbon catalyst.

Preferably, the catalyst is a platinum-activated carbon catalyst.

Preferably, the catalyst is a platinum-alumina catalyst.

Preferably, the catalyst is a platinum-calcium carbonate catalyst.

Preferably, the catalyst is a platinum-silica catalyst.

Preferably, the catalyst is a platinum-silica-aluminum catalyst.

Preferably, the catalyst is a copper-carbon catalyst.

Preferably, the catalyst is a copper-activated carbon catalyst.

Preferably, the catalyst is a copper-alumina catalyst.

Preferably, the catalyst is a copper-calcium carbonate catalyst.

Preferably, the catalyst is a copper-silica catalyst.

Preferably, the catalyst is a copper-silica-aluminum catalyst.

Preferably, the catalyst is a cobalt-carbon catalyst.

Preferably, the catalyst is a cobalt-activated carbon catalyst.

Preferably, the catalyst is a cobalt-alumina catalyst.

Preferably, the catalyst is a cobalt-calcium carbonate catalyst.

Preferably, the catalyst is a cobalt-silica catalyst.

Preferably, the catalyst is a cobalt-silica-aluminum catalyst.

Preferably, the catalyst is an iron-carbon catalyst.

Preferably, the catalyst is an iron-activated carbon catalyst.

Preferably, the catalyst is an iron-alumina catalyst.

Preferably, the catalyst is an iron-calcium carbonate catalyst.

Preferably, the catalyst is an iron-silica catalyst.

Preferably, the catalyst is an iron-silica-aluminum catalyst.

Preferably, the catalyst is a palladium catalyst and the palladium contained in the palladium catalyst is supported on a support material selected from carbon or activated carbon. Especially preferably, the palladium contained in the palladium catalyst is supported on activated carbon.

The terms “Pd/C catalyst” or “Pd/C” as used herein refer to palladium on carbon, which is a form of palladium that can be used as a heterogeneous hydrogenation catalyst and which is well known in the state of the art. Preferably, the palladium in the Pd/C catalyst or, respectively the Pd/C is supported on activated carbon, which thus functions as the support material. This results in a maximized surfaced area and activity. The skilled person knows how to prepare a Pd/C catalyst, which usually involves (i) combining a solution of palladium chloride and hydrochloric acid with an aqueous suspension of activated carbon followed by (ii) reducing the palladium (II) by the addition of formaldehyde.

Preferably, the catalyst, preferably the palladium catalyst, preferably the Pd/C catalyst, used in a method according to the present invention contains a total amount of the element of the catalyst, as described above, preferably palladium, of 25 wt.-% or less, preferably 22.5 wt.-% or less, particularly preferably 20 wt.-% or less, further preferably 17.5 wt.-% or less, more preferably 15 wt.-% or less, even further preferably 12.5 wt.-% or less, especially preferably 10 wt.-% or less, further preferably 7.5 wt.-% or less, even further preferably 6 wt.-% or less, based on to the sum of the weight of the element, preferably palladium, and the support material, preferably activated carbon. Particularly preferably, the catalyst, preferably the palladium catalyst, preferably the Pd/C catalyst used in a method according to the present invention contains a total amount of the element, preferably palladium, in range of from 5 wt.-% to 10 wt.-% based on the sum of the weight of the element, preferably palladium, and the support material, preferably activated carbon.

Typically, the catalyst, preferably the palladium catalyst, preferably the Pd/C catalyst, is provided as nominally 40 to 60% water wet, preferably as nominally 45 to 55% water wet, further preferably as nominally 50% water wet.

The term “solvent” as used herein refers to a substance that dissolves a solute, resulting in a solution. The quantity of solute that can dissolve in a specific volume of solvent varies with temperature. Preferably, the solvent as provided in step iii) of a method according to the present invention is a liquid under the conditions present during step iii). Preferably, the solvent used in step iii) of a method according to the present invention is a protic solvent.

Preferably in the mixture of water and one, two, three or more or all selected from methanol, ethanol, a 1,2-alkane diol with a total number of carbon atoms in the range of from 1 to 5, and a 1,3-alkane diol with a total number of carbon atoms in the range of from 1 to 5 in the preferred solvent provided in step iii), the weight ratio of water and methanol and/or ethanol and/or the 1,2-alkanediol(s) and/or the 1,3-alkane diol(s) is in a range of from 1:1 to 1:150, preferably 1:2 to 1:75, particularly preferably 1:4 to 1:40, wherein in case more than one of methanol, ethanol, 1,2-alkane diol(s) and 1,3-alkane diol(s) are present in the mixture, the combined weight of methanol, ethanol, 1,2-alkane diol(s) and 1,3-alkane diol(s), each as far as present, is considered for the weight ratio. The same applies accordingly, in case more than one of the 1,2-alkane diol(s) and/or more than one of the 1,3-alkane diol(s) is present.

The term “alkane diols with a total number of carbon atoms in the range of from 1 to 5”, as used herein, refers to alkane diols, wherein the alkane backbone has 1, 2, 3, 4 or 5 carbon atoms, for example a propane diol, a butane diol or a pentane diol. The alkane backbone may be linear or branched, preferably the alkane backbone is linear.

Preferably, the term “alkane diols with a total number of carbon atoms in the range of from 1 to 5”, as used herein, refers to 1,2-alkane diols, 1,3-alkane diols, or a mixture thereof. What was said above with regard to the number of carbon atoms applies accordingly.

Preferably, the solvent provided in step iii) of the method according to the invention is methanol or a mixture comprising methanol. Preferably said mixture includes water.

Preferably, the solvent provided in step iii) of the method according to the invention is ethanol or a mixture comprising ethanol. Preferably said mixture includes water.

Preferably, the solvent provided in step iii) of the method according to the invention is 1,2-propane diol or a mixture comprising 1,2-propane diol. Preferably said mixture includes water.

Preferably, the solvent provided in step iii) of the method according to the invention is 1,2-propane diol or a mixture comprising 1,3-propane diol. Preferably said mixture includes water.

It was surprisingly found that the solvent 2-propanol, which is used as the state of the art solvent in producing dihydrochalcones, only provided a low yield of hesperetin dihydrochalcone. However, changing the solvent to methanol, ethanol, alkane diols with a total number of carbon atoms in the range of from 1 to 5, or a mixture thereof led to an impressive 2.5-fold yield compared to the same reaction performed with 2-propanol (see Example 1 and 7). Similarly, using the solvents 1,2-propane diol or 1,3-propane diol (Examples 8 to 11) led to a much higher yield of hesperetin dihydrochalcone compared to 2-propanol (Example 7). Preferably, the formic acid provided in step iv) of a method according to the present invention has a purity of 50% or more, preferably of 70% or more, especially preferably of 75% or more, particularly preferably of 80% or more.

Preferably, reacting the formic acid provided in step iv) to a formate in step v) results in the following chemical equilibrium:

Formic acid+compound X⇄formate salt+compound Y

Preferably, the formate salt in the above chemical equilibrium includes the counter-ion provided by compound X.

Preferably, compound Y is selected from the group consisting of acetic acid, water, citric acid, tartaric acid, malic acid, propionic acid and mixtures thereof.

Preferably, the formate salt is selected from the group consisting of potassium formate, calcium formate, magnesium formate, ammonium formate, sodium formate and mixtures thereof, preferably selected from the group consisting of sodium formate, ammonium formate, and mixtures thereof. Especially preferably, the formate salt is sodium formate. Especially preferably, the formate salt is ammonium formate. Especially preferably, the formate salt is a mixture of sodium formate and ammonium formate.

The term “the formate salt is a mixture of sodium formate and ammonium formate” as used herein is to be understood such that both formates, sodium formate and ammonium formate, are present in the chemical equilibrium.

Preferably, compound X provides a counter-ion to the formate, which is selected from the group consisting of potassium ion, calcium ion, magnesium ion, ammonium ion, sodium ion and mixtures thereof.

Preferably, the formic acid provided in step iv) of a method according to the present invention is reacted in step v) with compound X, wherein compound X is selected from the group consisting of potassium acetate, calcium acetate, magnesium acetate, ammonium acetate, sodium acetate, sodium acetate trihydrate, potassium hydroxide, calcium hydroxide, magnesium hydroxide, ammonium hydroxide, sodium hydroxide, potassium citrate, calcium citrate, magnesium citrate, ammonium citrate, sodium citrate, potassium tartrate, calcium tartrate, magnesium tartrate, ammonium tartrate, sodium tartrate, potassium malate, calcium malate, magnesium malate, ammonium malate, sodium malate, potassium propanoate, calcium propanoate, magnesium propanoate, ammonium propanoate, sodium propanoate as well as their hydrates and mixtures thereof. Especially preferably, the formic acid provided in step iv) of a method according to the present invention is subsequently reacted with sodium acetate (i.e. compound X is sodium acetate), preferably sodium acetate trihydrate. Especially preferably, the formic acid provided in step iv) of a method according to the present invention is subsequently reacted with ammonium hydroxide (i.e. compound X is ammonium hydroxide). Especially preferably, the formic acid provided in step iv) of a method according to the present invention is subsequently reacted with ammonium hydroxide and with sodium acetate (i.e. compound X is a mixture of sodium acetate and ammonium hydroxide), preferably sodium acetate trihydrate.

The term “compound X is a mixture of sodium acetate and ammonium hydroxide” as used herein is to be understood such that compound X represents both, sodium acetate and ammonium hydroxide, which are thus both present in the chemical equilibrium.

Preferably, compound X is provided in excess, with regard to the amount of formic acid and the reaction for obtaining the respective formate. Preferably, compound X is provided in step v), wherein the molar ratio of compound X and of the formic acid provided in step iv) is in a range of from 10:11 to 10:50, preferably in a range of from 10:11 to 10:20, particularly preferably in a range of from 10:11 to 10:15.

It was surprisingly found that providing a formate via steps iv) and v), particularly in comparison to simply adding the corresponding formate to the further compounds, positively influenced the method according to the invention. Particularly, for the conversion of hesperetin to hesperetin dihydrochalcone in step vii) it is particularly beneficial to provide the formate via steps iv) and v).

When providing the formate via steps iv) and v), typically, not all of the formic acid is converted to the formate. Typically and preferably, the reaction results in a chemical equilibrium, in which both, the corresponding formate and formic acid are present, as described above. In this case, compound X is also present in the equilibrium. Preferably, compound X and the acetic acid (compound Y) provide a buffer, which buffers the pH in the method according to the invention, particularly when converting hesperetin to hesperetin dihydrochalcone in step vii). The provided buffer is particularly advantageous in case compound X is provided in excess or, respectively, in a molar ratio as described above.

Preferably, compound X is or comprises sodium acetate. In this case, a chemical equilibrium of formic acid and sodium acetate together with sodium formate is provided, in which an acetic acid/acetate buffer is provided. The same applies accordingly to other selections for compound X.

It was surprisingly found that the pH during the conversion of hesperetin to hesperetin dihydrochalcone in step vii) influences the quality of the reaction product. Thus, a buffered pH, as described above, advantageously increases the quality of the reaction product of the method according to the invention, i.e. less side-products are obtained.

Preferably, the catalyst, preferably the palladium catalyst, particularly preferably the Pd/C catalyst used in a method according to the present invention is removed from the reaction mixture in step viii) by means of filtration, preferably by means of a filtration via a filter plate a bag filter, kieselguhr or a combination of two or all thereof. Particularly preferably the filtration is a filtration via a filter plate and/or a bag filter.

The term “kieselguhr” as used herein refers to diatomaceous earth, which is also known as diatomite or kieselgur and which is a naturally occurring, soft, siliceous sedimentary rock that can be crumbled into a fine powder having a white to off-white colour.

Preferably, the catalyst, preferably the palladium catalyst, preferably the Pd/C catalyst used in a method according to the present invention is rinsed with water after being removed from the reaction mixture. Preferably, the catalyst, preferably the palladium catalyst, preferably he Pd/C catalyst used in a method according to the present invention can at least partially be re-used.

Preferably, the molar ratio of the hesperetin provided in step i) and the element of the catalyst as described above (i.e. palladium, ruthenium, gold, platinum, copper, cobalt or iron, preferably palladium) and provided in step ii) is in a range of from 8:1 to 75:1, preferably in a range of from 9:1 to 70:1, particularly preferably in a range of from 10:1 to 65:1, further preferably in a range of from 12:1 to 60:1, more preferably in a range of from 15:1 to 55:1, especially preferably in a range of from 20:1 to 50:1.

Preferably, the catalyst is a palladium catalyst and the molar ratio of the hesperetin provided in step i) and the palladium in the palladium catalyst provided in step ii) is in a range of from 8:1 to 75:1, preferably in a range of from 9:1 to 70:1, particularly preferably in a range of from 10:1 to 65:1, further preferably in a range of from 12:1 to 60:1, more preferably in a range of from 15:1 to 55:1, especially preferably in a range of from 20:1 to 50:1.

It is of particular advantage that in the method according to the invention, less of the element of the catalyst, such as palladium, is required. This facilitates the use of the obtained product for being further processed into a nutritional or pharmaceutical product. Furthermore, requiring less of the element, preferably palladium, provides a more environment-friendly and cost-efficient process, since these elements are very expensive. Particularly, palladium is the most expensive of the commercial noble metals.

Preferably, the molar ratio of the hesperetin provided in step i) and the formic acid provided in step iv) is in a range of from 1:1 to 1:6, preferably in a range of from 1:1.25 to 1:4, particularly preferably in a range of from 1:1.5 to 1:3.

Preferably, the molar ratio of the hesperetin provided in step i) and the formate resulting from the reaction in step v) is in a range of from 2:1 to 1:3.5, preferably in a range of from 1:1.25 to 1:3, particularly preferably in a range of from 1:1.5 to 1:2.5.

Furthermore, it is preferred that the molar ratio of the hesperetin provided in step i) and the solvent provided in step iii) is in a range of from 1:300 to 1:50, preferably in a range of from 1:220 to 1:90, particularly preferably in a range of from 1:210 to 1:100.

Advantageously, the method according to the invention requires less educts, such as formic acid, the formate or the solvent, and simultaneously provides a higher or at least comparable yield of hesperetin dihydrochalcone compared to the methods of the prior art.

Preferably, the method according to the present invention further comprises the step or the steps
ix) removing the solvent from the reaction mixture, preferably after step viii) is performed, and/or
x) adding water to the reaction mixture to effect the precipitation of hesperetin and/or hesperetin dihydrochalcone, and/or
xi) purification of hesperetin and/or hesperetin dihydrochalcone from the reaction mixture to obtain a purified mixture comprising hesperetin dihydrochalcone and optionally hesperetin.

The term “mixture comprising hesperetin dihydrochalcone and optionally hesperetin” as used herein describes a mixture comprising both, hesperetin and hesperetin dihydrochalcone or a mixture comprising hesperetin dihydrochalcone. These mixtures may comprise further components, such as (residual) water. However, the term also includes a mixture consisting of hesperetin and hesperetin dihydrochalcone or a mixture consisting of hesperetin dihydrochalcone.

Preferably, the solvent is at least partially removed from the reaction mixture in step ix) via reduced-pressure evaporation. Preferably, the pressure during reduced-pressure evaporation is 500 mbar or less, preferably, 400 mbar or less, preferably 300 mbar or less, especially preferably 200 mbar or less. Preferably, the temperature during reduced-pressure evaporation is in the range of from 30° C.-70° C., preferably 40° C.-65° C., especially preferably 50° C.-60° C.

Additionally or alternatively, the solvent may be removed from the reaction mixture in step ix) by distillation and/or membrane filtration.

The term “removing the solvent” as used herein describes a removal of the solvent, which may but does not need to be a complete removal of the solvent. It is preferred that in step ix) at least 30 wt.-% of the solvent, preferably at least 40 wt.-% of the solvent, particularly preferably at least 50 wt.-% of the solvent, especially preferably at least 55 wt.-% most preferably at least 60 wt.-% of the solvent, based on the total weight of the solvent provided in step iii) is removed. The skilled person knows how much solvent was provided in step iii) and thus knows how much solvent shall be removed.

The term “purification” as used herein describes a step of increasing the concentration of one or more substance(s) in a mixture of components. The step of increasing the concentration is typically achieved by removing other components of the mixture. Particularly, the term “purification” as used herein includes but does not require obtaining the isolated product(s).

Furthermore, it is preferred that in a method according to the present invention step x) is present. It is also preferred that in said step x), the pH value of the reaction mixture is adjusted to a pH value in the range of from 4 to 8, preferably in the range of from 5 to 7.5, particularly preferably in the range of from 6 to 7.

Adjusting the pH value to such a range is to be understood such that the pH value is determined and preferably such that an adjustment is performed only in case the determined pH value is outside the described range. Preferably, the adjustment of the pH value, if necessary or if performed, is performed with 10% sulfuric acid.

A pH value in such a range provides a particularly advantageous precipitation of hesperetin and/or hesperetin dihydrochalcone.

Furthermore, a method according to the present invention is preferred, wherein step xi) is present, preferably wherein the method also comprises one of step ix) or x), and
wherein the purification in step xi) comprises or consists of a filtration, preferably followed by one, two, three or more washing step(s), and/or
wherein step xi) further comprises drying the purified mixture to a defined water content.

Preferably, the step of filtration in step ix) is performed with a filter with a mesh size in the range of from 0.5 μm to 100 μm, preferably in the range of from 0.75 μm to 50 μm, further preferably in the range of from 1 μm to 10 μm.

Preferably, the filtration in step ix) is performed with a 10 μm filter plate. Additionally or alternatively it is preferred that the filtration in step ix) is performed with a bag filter with a 1 μm filter.

Preferably, the filtration in step xi) is performed with a bag filter with a 1 μm filter.

Preferably, the drying in step xi) is performed by a method selected from the group consisting of heat drying, drying with a stream of nitrogen, sun drying, hot air drying, combined air and heat pump drying, vacuum oven drying, freeze drying, spray drying, and (vacuum) belt drying.

Preferably, the step of drying in step xi) comprises two or more drying steps. Preferably, the step of drying comprises two or more drying steps with at least two methods selected from the group consisting of heat drying, drying with a stream of nitrogen, sun drying, hot air drying, combined air and heat pump drying, vacuum oven drying, freeze drying, spray drying, and (vacuum) belt drying.

Preferably, the water content is determined by dry-loss-method or Karl-Fischer Titration.

It is preferred that the water content of the purified mixture after the drying step(s) in step xi), as described above, is at most 70 wt.-%, preferably at most 60 wt.-%, particularly preferably at most 50 wt.-%, especially preferably at most 40 wt.-% more preferably at most 30 wt.-%, even further preferably at most 20 wt.-%, particularly preferably at most 10 wt.-%, most preferably at most 7.5 wt.-%, based on the total weight of the purified and dried mixture.

For a method according to the present invention it is preferred that in step vii) the reaction mixture is heated to a temperature in the range of from 35 to 50° C., preferably in the range of from 38 to 48° C.

It was found that heating the reaction mixture to a temperature in such a range provides a particularly advantageous conversion of hesperetin to hesperetin dihydrochalcone.

Preferably, in step vii) the temperature of the reaction mixture is maintained for a time in the range of from 30 to 360 minutes, preferably in the range of from 30 to 240 minutes, particularly preferably in the range of from 30 to 180 minutes. Preferably, the reaction mixture is stirred, preferably constantly stirred, while maintaining the temperature.

The term “maintaining the temperature” as used herein describes a process, in which the temperature is substantially maintained. Slight fluctuations of the temperature are still understood as “maintaining the temperature”. Preferably, the temperature is maintained at the set temperature ±5° C., preferably at the set temperature ±2.5° C.

It is also preferred that the conversion of hesperetin to hesperetin dihydrochalcone in step vii) is stopped before the total amount of hesperetin has been converted, preferably by removing the catalyst according to step viii).

It has been observed that stopping the conversion before the total amount of hesperetin has been converted and thus providing a mixture of hesperetin and hesperetin dihydrochalcone enhances the sweet modulating properties of hesperetin dihydrochalcone and caused a better mouthfeel (see Examples 12 to 14).

Moreover, it is preferred that the conversion of hesperetin to hesperetin dihydrochalcone is stopped such that the ratio of hesperetin and obtained hesperetin dihydrochalcone in the reaction mixture is in a range of from 1:1 to 1:2000, preferably in the range of from 1:1.25 to 1:1000, particularly preferably in the range of from 1:1.5 to 1:500, further preferably in the range of from 1:2 to 1:100, especially preferably in the range of from 1:3 to 1:50, even further preferably in the range of from 1:3.5 to 1:10.

The skilled person knows how much educts and catalyst are provided and used for the conversion. Based on literature values, the skilled person can calculate the time of the conversion, in which the respective amount of hesperetin is converted to hesperetin dihydrochalcone. Thus, the skilled person can calculate the time when to stop the reaction to obtain a ratio of hesperetin and hesperetin dihydrochalcone in the described range. Additionally or alternatively, a sample may be taken from the reaction mixture and be analysed via methods such as LC-MS to determine the conversion rate.

It was advantageously found that in a mixture including hesperetin and hesperetin dihydrochalcone in such a range, a synergistic effect on the sweetness-modulating properties of hesperetin dihydrochalcone was observed (see Examples 12 to 14).

Preferably, the ratio of hesperetin and obtained hesperetin dihydrochalcone in the reaction mixture is determined after step viii) is performed, and the ratio of hesperetin and obtained hesperetin dihydrochalcone in the reaction mixture is adjusted to a ratio in a range of from
1:1 to 1:2000, preferably in the range of from 1:1.25 to 1:1000, particularly preferably in the range of from 1:1.5 to 1:500, further preferably in the range of from 1:2 to 1:100, especially preferably in the range of from 1:3 to 1:50, even further preferably in the range of from 1:3.5 to 1:10,
preferably wherein the adjustment is performed only if the determined ratio is not within this range,
wherein the adjustment is performed by addition or removal of hesperetin and/or by addition or removal of hesperetin dihydrochalcone.

Preferably, the ratio of hesperetin and obtained hesperetin dihydrochalcone in the reaction mixture is adjusted in a way allowing hesperetin to synergistically enhance the sweetness-modulating properties of hesperetin dihydrochalcone, as described above.

The present invention further relates to a method for imparting or modifying a sweet taste impression, comprising the following steps:
(a) providing hesperetin dihydrochalcone by a method according to the invention,
(b) mixing the hesperetin dihydrochalcone obtained in step (a) with a substance or product, of which a sweet taste impression shall be imparted or modified.

The term “modifying a sweet taste impression” as used herein describes any kind of modification, such as optimizing or increasing a sweet taste impression.

Preferably, substances or products, of which a sweet taste impression shall be imparted or modified are selected from the group consisting of aliphatic flavouring substances, especially saturated aliphatic alcohols, such as ethanol, isopronanol, butanol, isoamyl alcohol, hexanol, 2-heptanol, octanol (1-/2-/3-), decanol, unsaturated aliphatic alcohols, such as cis-2 pentenol, cis-3 hexenol, trans-2 hexenol, trans-3 hexenol, cis-2 octenol, 1-octen-3-ol, cis-6 nonen-1-ol, trans-2, cis-6 nonadienol, aliphatic aldehydes such as saturated aliphatic aldehydes (e.g. acetaldehyde, propionaldehyde, butyraldehyde, isobutyraldehyde, valeraldehyde, isolvaleraldehyde, hexanal, 3-methyl hexanal, octanal, nonanal, or mono-or multi-unsaturated aliphatic aldehydes, such as 2-methyl but-2-enal, trans-2 hexenal, cis-3 hexenal, cis-4 hexenal, trans-2 octenal, trans-2 nonenal, cis-6 nonenal, trans-2, cis-6 nonadienal, trans 2 decenal, trans-2, trans-decadienal, aliphatic ketones, e.g. saturated ketones (such as 2-butanone, 2-pentanone, 2-heptanone, 2-octanone, 2-methylheptan-3-one, 2-decanone, 2-undecanone), unsaturated ketones (such as 1-penten-3-one, 1-hexen-3-one, 5-methyl-3-hexenone, 3-hepten-2-one, 1-octen-3-one, 2-octen-4-one, 3-octen-2-one, 3-none-2-one), aliphatic diketones and aliphatic diketoles, e.g. diacetyl, acetyl methyl carbinol, 2,3-hexanedione, aliphatic acids, such as straight-chain saturated acids, such as acetic acid, propionic acid, butyric acid, isobutyric acid, valeric acid, heptanoic acid, octanoic acid, decanoic acid, branched-chain saturated acids, such as 2-methyl heptanoic acid, 4-ethyl octanoic acid, and unsaturated acids, such as 2-butenoic acid, 2-pentenoic acid, 4-pentenoic acid, 2-methyl pentenoic acid, trans-3 hexenoic acid, cis-3 hexenoic acid, 3-octenoic acid, linoleic acid), aliphatic esters, such as saturated esters, e.g. methyl acetate, methylbutyrate, methyl-2-methylbutyrate, methyl hexanoate, ethylacetate, ethylbutyrate, ethyl-2-methylbutyrate, ethyl-3-methylbutyrate, ethyl hexanoate, ethyl decanoate, isopropyl acetate, isobutyl acetate, isobutyl valerate, isoamyl acetate, isoamyl butyrate, isoamyl isovalerate, hexyl acetate, hexyl hexanoate, 3-octyl acetate and unsaturated esters, such as methyl 2-hexenoate, allyl hexanoate, cis-3 hexenyl acetate, cis-3 hexenyl butyrate, aliphatic thiols and dithiols (e.g. propane thiol, allyl mercaptan, 1-methoxy-3-methylbutane-3-thiol, dimethyl sulfide, dimethyl trisulfide, dipropyl sulfide, diallyl trisulfide, other aliphatic sulfur compounds, such as 2-mercapto-3-butanol, methyl thio propanal, 3-mercapto-pentanone, 4-methoxy-2-methyl-2-mercaptobutanone, methyl thiobutyrate, methyl thiobutyrate, methyl 3-methylthiopropionate, aliphatic nitrogen compounds, such as butyl amine, trimethyl amine, allyl isothiocyanate, isopropyl isothiocyanate, alicyclic compounds, such as alicyclic ketones, e.g. cis-jasmone, isophorone, 4-ketoisophorone, alicyclic esters such as methyl jasmonate, hedione, terpenes, e.g. terpene alcohols, such as linalool, citronellol, geraniol, nerol, alpha terpineol, menthol, 8-p-menthene-1,2-diol, fenchol, borneol, nerolidol, hotrienol, terpene aldehydes such as geranial, neral, citronellal, beta-sinensal, terpene ketones, such as alpha-ionone, (D)-carvone, (L)-carvone, nootkatone, piperitone, menthone, alpha damascone, beta damascene, damascenone, terpene esters, such as linalyl acetate, geranyl acetate, citronellyl actetate, carvyl acetate, fenchyl acetate, terpene sulphur compounds, 4-mentha-8-thiol-3-one, thiogeraniol, para-menth-1-ene-8-thiol, mercapto p-menthan-3-one, terpene hydrocarbons, such as D-limonene, L-limonene, alpha-pinene, beta-pinene, ocimene, alpha-terpinene, gamma-terpinene, beta-bisabolene, valencene, terpene oxides, such as 1,8-cineole, rose oxide, mint lactone, menthofuran, aromatic compounds, e.g. aromatic alcohols, such as benzyl alcohol, cinnamyl alcohol, 2-phenyl alcohol, aromatic aldehydes, such as benzaldehyde, cinnamic aldehyde, 5-methyl-2-phenylhexenal, salicylaldehyde, 4-hydroxy benzaldehyde, cyclamen aldehyde, 2-phenyl-2-butenal, aromatic acids, such as 2-phenyl acetic acid, cinnamic acid, aromatic esters such as benzyl acetate, benzyl salicylate, anisyl acetate, methyl phenyl acetate, methyl benzoate, methyl salicylate, methyl cinnamate, aromatic phenols, such as phenol, ortho-cresol, para-cresol, 2,3-dimethyl phenyl, 2-ethyl phenol, 2,3,5-trimethyl phenol, 4-vinyl phenol, guaiacol, 4-vinyl guaiacol, eugenol, thymol, carvacrol, aromatic sulphur compounds, such as thiophenol, diphenyl disulphide, aromatic nitrogen compounds, such as methyl anthranilate, methyl N-methyl anthranilate, aromatic ethers such as vanillin, ethylvanillin, anethol, aromatic oxides, such as heliotropine, diphenyl oxide, aromatic lactones, such as coumarin, dihydro coumarin, heterocyclic compounds, such as heterocyclic lactones, e.g. gamma butyrolactone, gamma-nonalactone, gamma decalactone, delta decalactone, jasmin lactone, delta dodecalactone, ambrettolide, heterocyclic furanes, such as furfuryl alcohol, furfural, 2-acetyl furan, theaspirane, 2-methyl tetrahydro furan-3-one, furfuryl mercaptane, 2-methyl 3-furanthiol, 2-methyl 3-tetrahydro furanthiol, difurfuryl sulfide, difurfuryl disulfide, heterocyclic pyrans, such as maltol, ethyl maltole, rose oxide, maltol isobutyrate, heterocyclic pyrroles such as indole, 2-acetyle pyrrole, pyrrolidine, heterocyclic pyrazines, such as 2-methyl pyrazine, 2,3-dimethyl pyrazine, 2-methyl 3-ethyl pyrazine, trimethyl pyrazine, 2-acetyl pyrazine, 2-methoxy 3-methyl pyrazine, 2-methoxy 3-ethyl pyrazine, 2-methoxy 3-isobutyl pyrazine, 2-ethyl 3-methylthio pyrazine, heterocyclic thiazoles, such as thiazole, 2-methyl thiazole, 4-methyl 5-vinyl thiazole, 2-isobutyl thiazole, 2-acetyl thiazole.

Preferably, substances or products, of which a sweet taste impression shall be imparted or modified can also be selected from the group consisting of flavouring raw materials and flavouring preparations, e.g. essential oils, concretes, absolutes, extract or tinctures from raw materials such as citrus (e.g. lemon, lime, mandarine, bergamotte, grapefruit bitter orange, peel or essence oils), herbs (dill, parsley, cumin, rosemary, sage, clary sage, basil, tarragon, thyme, oregano, savoury, majoram, all spice, mace, nutmeg, clove leave, clove bud, caraway, cinnamom leaves, cinnamom bark, cassia, cardamom, ginger, galangal, turmeric, coriander seed, coriander leaf, fenugreek, juniper berry, wormwood, laurel leaves, eucalyptus, white pepper, green pepper, white pepper, carrot seed, celery seed, lovage leaf, asa foetida, onion, leek, garlic, mustard, horse radish, capsicum, paprika, sea weed, valerian oil, fir needle, spearmint, peppermint, wintergreen, buchu leaf, black currant buds, fennel, star anise, jambu, long pepper, davana, orris, mimosa, cassie, violet leaves, ho leaf, jasmin, ylang ylang, cananga, osmanthus, angelica, clary sage, ambrette seed, hops, camomile, lavender, rose, geranium, citronella, palmarosa, litsea cubeba, lemon grass, tagetes, neroli, petitgrain, mate, cognac oil, coffee, cola nut, cocoa, green tea, black tea, white tea, gentian, tolu balm, benzoe resin, peru balm, cascarilla, galbanum, vetiver, labdanum, patchouli, sandalwood, cedarwood, guaiac wood, oak wood, massoi bark, vanilla pods, tonka bean, as well as enriched fractions thereof.

Furthermore, substances or products, of which a sweet taste impression shall be imparted or modified can additionally or alternatively be selected from the group consisting of juice concentrates, such as orange juice, lemon juice, strawberry, cherry juice, or passion fruit juice concentrates, waterphases and recoveries from raw materials such as citrus (lemon, lime, orange, mandarine, grapefruit), apple, pear, quince, mispel, red fruits (raspberry, strawberry, blueberry, blackberry, Amellanchia (June plum), rose hip, cranberry, plum, prune, red and black currant, etc.) yellow fruits (peach, apricot, nectarine, banana, etc.), tropical fruits (mango, passionfruit, pineapple, lychee, etc.), vegetables (e.g. cucumber, tomato) and spices (e.g. ginger), acetophenone, allyl caproate, alpha-ionone, beta-ionone, anisaldehyde, anisyl acetate, anisyl formate, benzaldehyde, benzothiazole, benzyl acetate, benzyl alcohol, benzyl benzoate, beta-ionone, butyl butyrate, butyl caproate, butylidene phthalide, carvone, camphene, caryophyllene, cineol, cinnamyl acetate, citral, citronellol, citronellal, citronellyl acetate, cyclohexyl acetate, cymene, damascone, decalactone, dihydrocoumarin, dimethyl anthranilate, dodecalactone, ethoxyethyl acetate, ethylbutyric acid, ethyl butyrate, ethyl caprate, ethyl caproate, ethyl crotonate, ethylfuraneol, ethylguaiacol, ethylisobutyrate, ethylisovalerate, ethyl lactate, ethylmethyl butyrate, ethyl propionate, eucalyptol, eugenol, ethyl heptylate, 4-(p-hydroxyphenyl)-2-butanone, gamma-decalactone, geraniol, geranyl acetate, geranyl acetate, grapefruit aldehyde, methyl dihydrojasmonate (e.g. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl caproate, trans-2-hexenyl caproate, cis-3-hexenyl formate, cis-2-hexyl acetate, cis-3-hexyl acetate, trans-2-hexyl acetate, cis-3-hexyl formate, para-hydroxybenzyl acetone, isoamyl alcohol, isoamyl isovalerate, isobutyl butyrate, isobutyraldehyde, isoeugenol methyl ether, isopropyl methylthiazole, lauric acid, levulinic acid, linalool, linalool oxide, linalyl acetate, menthol, menthofuran, methyl anthranilate, methylbutanol, methylbutyric acid, 2-methylbutyl acetate, methyl caproate, methyl cinnamate, 5-methylfurfural, 3,2,2-methylcyclopentenolone, 6,5,2-methylheptenone, methyl dihydrojasmonate, methyl jasmonate, 2-methylmethyl butyrate, 2-methyl-2-pentenol acid, methylthiobutyrate, 3,1-methylthiohexanol, 3-methylthiohexyl acetate, nerol, nerol acetate, trans, trans-2,4-nonadienal, 2,4-nonadienol, 2,6-nonadienol, 2,4-nonadienol, nootkatone, delta-octalactone, gamma-octalactone, 2-octanol, 3-octanol, 1,3-octenol, 1-octyl acetate, 3-octyl acetate, palmitic acid, paraldehyde, phellandrene, pentanedione, phenylethyl acetate, phenylethyl alcohol, phenylethyl isovalerate, piperonal, propionaldehyde, propyl butyrate, pulegone, pulegol, sinensal, sulfurol, terpinene, terpineol, terpinolene, 8,3-s thiomenthanone, 4,4,2-thiomethylpentanone, thymol, delta-undecalactone, gamma-undecalactone, valencene, valeric acid, vanillin, acetoin, ethylvanillin, ethylvanillin isobutyrate (=3-ethoxy-4-isobutyryloxybenzaldehyde), 2,5-dimethyl-4-hydroxy-3 (2H)-furanone and derivatives thereof (here preferably homofuraneol (=2-ethyl-4-hydroxy-5-methyl-3 (2H)-furanone), homofuronol (=2-ethyl-5-methyl-4-hydroxy-3 (2H)-furanone and 5-ethyl-2-methyl-4-hydroxy-3 (2H)-furanone), maltol and maltol derivatives (here preferably ethyl maltol), coumarin and coumarin derivatives, gamma-lactones (here preferably gamma-undecalactone, gamma-nonalactone, gamma-decalactone), delta-lactones (here preferably 4-methyldeltadecalactone, massoilactone, deltadecalactone, tuberolactone), methyl sorbate, divanillin, 4-hydroxy-2 (or 5)-ethyl-5 (or 2)-methyl-3 (2H) furanone, 2-hydroxy-3-methyl-2-cyclopentenone, 3-hydroxy-4,5-dimethyl-2 (5H)-furanone, acetic acid isoamyl ester, butyric acid ethyl ester, butyric acid-n-butyl ester, butyric acid isoamyl ester, 3-methyl-butyric acid ethyl ester, n-hexanoic acid ethyl ester, n-hexanoic acid allyl ester, n-hexanoic acid-n-butyl ester, n-octanoic acid ethyl ester, ethyl-3-methyl-3-phenylglycidate, ethyl-2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio) furan, 2-methyl-3-furanthiol, bis (2-methyl-3-furyl) disulphide, furfurylmercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3 (2H)-furanone, guaiacol, 3-hydroxy-4,5-dimethyl-2 (5H)-furanone, 3-hydroxy-4-methyl-5-ethyl-2 (5H)-furanone, cinnamaldehyde, cinnamon alcohol, methyl salicylate, isopulegol and (here not explicitly stated) stereoisomers, enantiomers, positional isomers, diastereomers, cis/trans isomers or epimers of these substances.

Additionally or alternatively, substances or products, of which a sweet taste impression shall be imparted or modified can also be selected from the group consisting of compounds or compound mixtures conveying a sweet taste, such as natural sweeteners, preferably naturally occurring sweet tasting substances, including plant extracts, such as sweet tasting carbohydrates (such as sucrose, trehalose, lactose, maltose, melizitose, melibiose, raffinose, palatinose, lactulose, D-fructose, D-glucose, D-galactose, I-rhamnose, D-sorbose, D-mannose, D-tagatose, D-arabinose, I-arabinose, D-ribose, D-glyceraldehyde, maltodextrin), sugar alcohols (such as erythritol, threitol, arabitol, ribitol, xylitol, sorbitol, mannitol, maltitol, isomaltit, dulcitol, lactitol), proteins (such as miraculin, pentaidin, monellin, thaumatin, curculin, brazzein, mabinlin), D-amino acids (such as D-phenylalanine, D-tryptophan) or extracts or fractions obtained from natural sources containing these amino acids and/or proteins and the physiologically acceptable salts of these amino acids and/or proteins, particularly the sodium, potassium, calcium or ammonium salts thereof; neohesperidindihydrochalkon, naringindihydrochalkon, steviolgylcoside, stevioside, steviolbiosid, rebaudioside, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A, cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, trans-Anethol, trans-cinnamaldehyd, bryoside, bryonoside, bryonodulcoside, carnosifloside, scandenoside, gypenoside, hematoxylin, cyanin, chlorogensäure, albiziasaponin, telosmoside, gaudichaudiosid, mogrosides, such as mogroside V, hernandulcine, monatin, glycyrrhetinic acid and its derivatives, particularly glycyrrhizin, preferably glycyrrhizin ammonium salt; extracts or enriched fractions of such extracts such as extracts of thaumatococcus or stevia ssp., particularly stevia rebaudiana, swingle extracts, particularly momordica or siratio grosvenoriior Luo-Han-Guo, extracts of glycerrhyzia ssp., particularly glycerrhyzia glabra, extracts of rubus ssp., particularly rubus suavissimus, extracts of lippia dulcis, extracts of mycetia balansae, preferably comprising balansin A and/or balansin B.

Additionally or alternatively, substances or products, of which a sweet taste impression shall be imparted or modified can also be selected from the group consisting of compounds or compound mixtures conveying a sweet taste, such as synthetic sweeteners, preferably synthetic sweet tasting substances, preferably selected from the group consisting of magap, sodium cyclamate or other physiologically acceptable salts of cyclamic acid, acesulfam K; neohesperidindihydrochalcone, naringindihydrochalcone, saccharin, saccharin sodium salt, aspartam, superaspartam, neotam, alitam, advantam, perillartin, sucralose, lugduname, carrelame, sucrononate or sucrooctate or mixtures thereof.

It is preferred that in the method for imparting or modifying a sweet taste impression according to the invention, in step (a) the hesperetin dihydrochalcone is provided by a method according to the invention, which comprises at least one of steps ix), x) or xi). Preferably the hesperetin dihydrochalcone is provided by a method according to the invention, which comprises step ix) and optionally steps x) and or xi). What was said with regard to these or other features of a method according to the invention, particularly with regard to preferred features, applies accordingly.

The invention is further characterized by illustrative, non-limiting examples.

### EXAMPLES

The amounts of several components in the following examples are indicated as equivalents (“eq.”), which describe molar ratio of said component and a basis. If not stated otherwise, the indicated equivalents refer to hesperetin as a basis. Thus, using 2.00 eq. of formic acid is to be understood that for each mol of the basis (e.g. hesperetin), 2.00 mol of formic acid are used.

The purity of the used compounds is assumed as 100% unless otherwise stated.

### Example 1

First, 600 mL of ethanol were transferred into a 1-L three-necked flask and under ice cooling 10.3 mL of an ammonium hydroxide solution (25% aqueous, 1 eq.) were added. Subsequently, the solution was cooled while stirring and 6.4 mL formic acid (80%, 132.4 mmol, 2.00 eq.) were slowly dripped into the solution. The mixture was stirred for 30 minutes. Then, 20 g of hesperetin (66.2 mmol, 1 eq.) were added and 9 g of Pd/C (5 wt.-%, nominally 50% water wet, 2.11 mmol of Pd) were mixed into the solution. Then, the reaction mixture was heated to 47° C. within 29 min. The reaction mixture was stirred at this temperature for one hour. After cooling to room temperature (RT), the catalyst was filtered off via kieselguhr and then the catalyst was rinsed with 200 mL of water. The ethanol was removed via reduced-pressure evaporation. Subsequently, 600 ml of water were added to the filtrate and while stirring the pH was adjusted to 6.5 employing sulphuric acid (10%). Then, the reaction mixture was stirred overnight. Afterwards, the reaction mixture was filtered and the obtained solid was washed with water and dried overnight at 40° C. in a vacuum drying oven. 19.22 g of a beige coloured solid were obtained. The residual water content was determined to be 4.06%. Quantification was performed by means of HPLC. The results are shown in Table 1.

### Example 2

First, 200 mL of ethanol were transferred into a 1-L three-necked flask and under ice cooling 5.2 mL of an ammonium hydroxide solution (25% aqueous, 1 eq.) were added. Subsequently, the solution was cooled while stirring and 3.2 mL formic acid (80%, 66.2 mmol, 2.00 eq.) were slowly dripped into the solution. The mixture was stirred for 30 minutes. Then, 10 g of hesperetin (33.1 mmol, 1 eq.) were added and 4.5 g of Pd/C (5 wt.-%, nominally 50% water wet, 1.06 mmol of Pd) were mixed into the solution. Then, the reaction mixture was heated to 45° C. and stirred at this temperature for 120 min. After cooling to RT, the catalyst was filtered off via kieselguhr and then the catalyst was rinsed with 100 ml of water. The ethanol was removed via reduced-pressure evaporation. Subsequently, 300 ml of water were added to the filtrate and while stirring the pH was adjusted to 6.5 employing sulphuric acid (10%). Then, the reaction mixture was stirred overnight. Afterwards, the reaction mixture was filtered and the obtained solid was washed with water and dried overnight at 40° C. in a vacuum drying oven. 10 g of a beige coloured solid were obtained. The residual water content was determined to be 3.58%. Quantification was performed by means of HPLC. The results are shown in Table 1.

### Example 3

First, 100 mL of ethanol were transferred into a 0.5-L three-necked flask and under ice cooling 5.2 mL of an ammonium hydroxide solution (25% aqueous, 1 eq.) were added. Subsequently, the solution was cooled while stirring and 3.2 mL formic acid (80%, 66.2 mmol, 2.00 eq.) were slowly dripped into the solution. The mixture was stirred for 30 minutes. Then, 10 g of hesperetin (33.1 mmol, 1 eq.) were added and 4.5 g of Pd/C (5 wt.-%, nominally 50% water wet, 1.06 mmol of Pd) were mixed into the solution. Then, the reaction mixture was heated to 45° C. and stirred at this temperature for 120 min. After cooling to RT, the catalyst was filtered off via kieselguhr and then the catalyst was rinsed with 100 ml of water. The ethanol was removed via reduced-pressure evaporation. Subsequently, 300 ml of water were added to the filtrate and while stirring the pH was adjusted to 6.5 employing sulphuric acid (10%). Then, the reaction mixture was stirred overnight. Afterwards, the reaction mixture was filtered and the obtained solid was washed with water and dried overnight at 40° C. in a vacuum drying oven. 8.75 g of a beige coloured solid were obtained. The residual water content was determined to be 4.36%. Quantification was performed by means of HPLC. The results are shown in Table 1.

### Example 4

As a first step, the ammonium formate was produced in a separate flask. For this, 5.2 mL of an ammonium hydroxide solution (25% aqueous, 1 eq.) were transferred into a flask and 3.2 mL formic acid (80%, 66.2 mmol, 2.00 eq.) were slowly mixed into the solution while cooling on ice. The reaction mixture was stirred for 30 min and subsequently heated to RT.

In a separate flask, 10 g of hesperetin (33.1 mmol, 1 eq.) were dissolved in 300 ml of ethanol and mixed with 3 g of Pd/C (5 wt.-%, nominally 50% water wet, 0.70 mmol of Pd). The reaction mixture was slowly heated to 45° C. Then, the ammonium formate solution was slowly added to the reaction mixture over the course of 30 min. The mixture was stirred for 90 min at 45° C. After cooling to RT, the catalyst was filtered off via kieselguhr and then the catalyst was rinsed with 100 ml of water. Further 100 ml of water were added to the filtrate. The ethanol was removed via reduced-pressure evaporation. Further 400 ml of water were added to the filtrate. Formation of a solid was already visible. The pH was adjusted to 7.1-7.2 employing sulphuric acid (10%). Then, the reaction mixture was stirred overnight. Afterwards, the reaction mixture was filtered and the obtained solid was washed with water and dried overnight at 40° C. in a vacuum drying oven. 9.34 g of a beige coloured solid were obtained]. NMR data showed that 11% of the solid were hesperetin and 89% of the solid were hesperetin dihydrochalcone. The residual water content was determined to be 3.63%. Additional quantification was performed by means of HPLC. The results are shown in Table 1.

### Example 5

As a first step, the ammonium formate was produced. For this, 3.2 g of ammonium carbonate (30% NH₃; 1 eq.) were transferred into a flask and mixed with 400 mL of ethanol. 3.2 mL formic acid (80%, 66.2 mmol, 2.00 eq.) were slowly mixed into the solution. The solution was stirred for 30 min at RT.

Then, 10 g of hesperetin (33.1 mmol, 1 eq.) were added to the solution and 4 g of Pd/C (5 wt.-%, nominally 50% water wet, 0.94 mmol of Pd) were step by step added to the solution. Formation of a gas was observed. After gas formation had decreased, the reaction mixture was slowly heated to 45° C. The mixture was stirred for 120 min at 45° C. After cooling to RT, the catalyst was filtered off via kieselguhr and then the catalyst was rinsed with 100 ml of water. Further 100 mL of water were added to the filtrate. The ethanol was removed via reduced-pressure evaporation. The residue obtained was mixed with 400 ml of water and the pH was adjusted to 7.1-7.2 employing sulphuric acid (10%). Then, the reaction mixture was stirred overnight at RT. Afterwards, the reaction mixture was filtered and the obtained solid was washed with water and dried overnight at 40° C. in a vacuum drying oven. 9.37 g of a colourless solid were obtained. Quantification was performed by means of HPLC and the residual water content was determined. The residual water content was determined to be 4.19%. Quantification was performed by means of HPLC. The results are shown in Table 1.

### Example 6

20 g of hesperetin (96%, 63.5 mmol, 1 eq.) were dissolved in 300 g ethanol and the solution was vigorously stirred under an inert gas atmosphere. Then, 11.9 g Pd/C (5 wt.-%, nominally 50% water wet, 2.7 mmol of Pd, 0.04 eq.) were added to the solution. At 40° C., a solution of 22.5 g sodium acetate trihydrate (165 mmol, 2.59 eq.), 7.5 g of formic acid (80%, 130 mmol, 2.04 eq.) and 27.5 g of water was added over the course of 60 min. Then, the reaction mixture was stirred for 120 min at a constant temperature of 40° C.±2° C.

The Pd/C was filtered off via a suction filter (5-10 μm pore size), which was rinsed with 125 ml of water. The obtained filtrate had a yellowish/orange colour and it was concentrated to half of the original volume via reduced-pressure evaporation (50-60° C., 200 mbar). Then, the mixture was diluted with 375 ml of water and cooled to 20° C. over the course of 120 min. The mixture was filtered and the filter cake was washed twice with 25 ml of water. The obtained solid was dried on the filter in a stream of nitrogen. The residual water content was determined to be about 50%. Quantification was performed by means of HPLC. The results are shown in Table 1.

### Example 7 (Comparative Example)

600 mL of 2-propanol were transferred into a 1-L three-necked flask and under ice cooling 10.3 mL of a ammonium hydroxide solution (25% aqueous, 1 eq.) were added. Subsequently, the solution was cooled while stirring and 6.4 mL formic acid (80%, 132.4 mmol, 2.00 eq.) were slowly dripped into the solution. The mixture was stirred for 30 minutes. Then, 20 g of hesperetin (66.2 mmol, 1 eq.) were added and 9 g of Pd/C (5 wt.-%, nominally 50% water wet, 2.11 mmol of Pd) were mixed into the solution. Then, the reaction mixture was heated to 47° C. within 29 min. The reaction mixture was stirred at this temperature for one hour. After cooling to room temperature (RT), the catalyst was filtered off via kieselguhr and then the catalyst was rinsed with 200 ml of water. The 2-propanol was removed via reduced-pressure evaporation. Subsequently, 600 mL of water were added to the filtrate and while stirring the pH was adjusted to 6.5 employing sulphuric acid (10%). Then, the reaction mixture was stirred overnight. Afterwards, the reaction mixture was filtered and the obtained solid was washed with water and dried overnight at 40° C. in a vacuum drying oven. 17.96 g of a beige coloured solid were obtained. The residual water content was determined to be 5.02%. Quantification was performed by means of HPLC. The results are shown in Table 1.

### Example 8

20 g of hesperetin (96%, 63.5 mmol, 1 eq.) were dissolved in 300 g 1,3-propane diol and the solution was vigorously stirred under an inert gas atmosphere. Then, 5.98 g Pd/C (5 wt.-%, nominally 50% water wet, 1.35 mmol of Pd, 0.02 eq.) were added to the solution. At 40° C., a solution of 22.5 g sodium acetate trihydrate (165 mmol, 2.59 eq.), 7.5 g of formic acid (80%, 130 mmol, 2.04 eq.) in 27.5 g of water was added over the course of 60 min. Then, the reaction mixture was stirred for 180 min at a constant temperature of 40° C.±2° C.

The Pd/C was filtered off via a suction filter (5-10 μm pore size). The obtained filtrate had a yellowish/orange colour and it was concentrated to 20% of the original volume via reduced-pressure evaporation (80-100° C., 3 mbar). Then, the mixture was diluted with 400 ml of water and cooled to 20° C. over the course of 160 min. The mixture was filtered and the filter cake was washed twice with 30 ml of water. The obtained solid was dried on the filter in a stream of nitrogen and dried in vacuum. The residual water content was determined to be about 4-5%. Quantification was performed by means of HPLC. The results are shown in Table 1.

### Example 9

20 g of hesperetin (96%, 63.5 mmol, 1 eq.) were dissolved in 300 g 1,2-propane diol and the solution was vigorously stirred under an inert gas atmosphere. Then, 5.98 g Pd/C (5 wt.-%, nominally 50% water wet, 1.35 mmol of Pd, 0.02 eq.) were added to the solution. At 40° C., a solution of 22.5 g sodium acetate trihydrate (165 mmol, 2.59 eq.), 7.5 g of formic acid (80%, 130 mmol, 2.04 eq.) in 30 g of water was added over the course of 60 min. Then, the reaction mixture was stirred for 180 min at a constant temperature of 40° C.±2° C.

The Pd/C was filtered off via a suction filter (5-10 μm pore size). The obtained filtrate had a yellowish/orange colour and it was concentrated to 20% of the original volume via reduced-pressure evaporation (80-100° C., 3 mbar). Then, the mixture was diluted with 400 mL of water and cooled to 20° C. over the course of 160 min. The mixture was filtered and the filter cake was washed twice with 30 ml of water. The obtained solid was dried on the filter in a stream of nitrogen and dried in vacuum. The residual water content was determined to be about 6-7%. Quantification was performed by means of HPLC. The results are shown in Table 1.

### Example 10

20 g of hesperetin (96%, 63.5 mmol, 1 eq.) were dissolved in 300 g 1,3-propane diol and the solution was vigorously stirred under an inert gas atmosphere. Then, 5.98 g Pd/C (10 wt.-%, nominally 50% water wet, 2.7 mmol of Pd, 0.04 eq.) were added to the solution. At 40° C., a solution of 22.5 g sodium acetate trihydrate (165 mmol, 2.59 eq.), 7.5 g of formic acid (80%, 130 mmol, 2.04 eq.) in 27.5 g of water was added over the course of 60 min. Then, the reaction mixture was stirred for 120 min at a constant temperature of 40° C.±2° C.

The Pd/C was filtered off via a suction filter (5-10 μm pore size). The obtained filtrate had a yellowish/orange colour and it was concentrated to 20% of the original volume via reduced-pressure evaporation (80-100° C., 3 mbar). Then, the mixture was diluted with 400 mL of water and cooled to 20° C. over the course of 160 min. The mixture was filtered and the filter cake was washed twice with 30 mL of water. The obtained solid was dried on the filter in a stream of nitrogen and dried in vacuum. The residual water content was determined to be about 4%. Quantification was performed by means of HPLC. The results are shown in Table 1.

### Example 11

20 g of hesperetin (96%, 63.5 mmol, 1 eq.) were dissolved in 300 g 1,3-propane diol and the solution was vigorously stirred under an inert gas atmosphere. Then, 5.98 g Pd/C (5 wt.-%, nominally 50% water wet, 1.35 mmol of Pd, 0.02 eq.) were added to the solution. At 40° C., a solution of 22.5 g sodium acetate trihydrate (165 mmol, 2.59 eq.), 7.5 g of formic acid (80%, 130 mmol, 2.04 eq.) in 27.5 g of water was added over the course of 60 min. Then, the reaction mixture was stirred for 180 min at a constant temperature of 40° C.±2° C.

The Pd/C was filtered off via a suction filter (5-10 μm pore size), which was rinsed with 200 ml of water. The obtained filtrate had a yellowish/orange colour and it was diluted with 1000 ml of water and cooled to 20° C. over the course of 160 min. The mixture was filtered and the filter cake was washed twice with 100 ml of water. The obtained solid was dried on the filter in a stream of nitrogen and dried in vacuum. The residual water content was determined to be about 6% and residual propane-1,3-diol content was determined to be about 7%. Quantification was performed by means of HPLC. The results are shown in Table 1.

It was surprisingly found that the solvent 2-propanol, which is commonly used in the prior art to produce other dihydrochalcones than hesperetin dihydrochalcone, lead to only a small yield of hesperetin dihydrochalcone. However performing the same experiment with ethanol (Example 1) led to a 2.5-fold yield of hesperetin dihydrochalcone compared to 2-propanol (Example 7). Similarly, using the solvents 1,2-propane diol or 1,3-propane diol (Examples 8 to 11) led to a much higher yield of hesperetin dihydrochalcone compared to 2-propanol (Example 7).

TABLE 1
Experimental results regarding the respective yields of hesperetin (HT)
and hesperetin dihydrochalcone (HC), as well as the ratio of the obtained
HT and HC and the ratio of used HT and Pd in the Pd/C catalyst

| | Example | | | | | | |
|---|---|---|---|---|---|---|---|
| Product | 1 | 2 | 3 | 4 | 5 | 6 | 7 |
| HC | 87.6% | 87.4% | 68.4% | 77.3% | 72.7% | 76% | 34.8% |
| HT | 4.5% | 4.1% | 10.6% | 9.9% | 16% | 11% | 48.7% |
| HC:HT | 19.47:1 | 21.32:1 | 6.45:1 | 7.81:1 | 4.5:1 | 6.9:1 | 0.71:1 |
| HT:Pd | 31.3:1 | 31.3:1 | 31.3:1 | 47:1 | 35.2:1 | 22.7:1 | 31.3:1 |

| | Example | | | |
|---|---|---|---|---|
| Product | 8 | 9 | 10 | 11 |
| HC | 91.2% | 90.7% | 89.7% | 82.9% |
| HT | 0% | 0% | 0% | 0% |
| HC:HT | — | — | — | — |
| HT:Pd | 47:1 | 47:1 | 23:1 | 47:1 |

Even though some of the examples lead to comparable yields as the method of Krishnamurty et al. (2006), it is to be noted that in these examples only a very small amount of the catalyst Pd/C and only small amounts of the further educts were used but though, comparable yields were obtained.

### Example 12

### Methanol

10 g of hesperetin (96%, 31.8 mmol, 1 eq.) were dissolved in 150 g methanol and the solution was vigorously stirred under an inert gas atmosphere. Then, 5.98 g Pd/C (5 wt.-%, nominally 50% water wet, 1.40 mmol of Pd, 0.02 eq.) were added to the solution. At 40° C., a solution of 11.25 g sodium acetate trihydrate (82.5 mmol, 2.59 eq.), 3.75 g of formic acid (80%, 65 mmol, 2.04 eq.) and 13.8 g of water was added over the course of 60 min. Then, the reaction mixture was stirred for 120 min at a constant temperature of 40° C.±2° C.

The Pd/C was filtered off via a suction filter (5-10 μm pore size), which was rinsed with 63 mL of water. The obtained filtrate was concentrated via reduced-pressure evaporation. Then, the mixture was cooled and diluted with 188 mL of water. The pH value is set to 6-7 if necessary. Then, the reaction mixture was stirred overnight at RT. Afterwards, the reaction mixture was filtered and the obtained solid was washed with water (2×25 mL) and dried overnight at 40° C. in a vacuum drying oven. 8.99 g (84.3% HC, 0.2% HT) of a beige solid were obtained. The residual water content was determined to be 4.09%. Quantification was performed by means of HPLC.
Yield HC: 78.3%
Yield HT: 0.19%
HC:HT 412.1:1
HT:Pd 22.7:1

### Example 13

### 1,2-Butane Diol

20 g of hesperetin (96%, 63.5 mmol, 1 eq.) were dissolved in 300 g 1,2-butane diol and the solution was vigorously stirred under an inert gas atmosphere. Then, 5.98 g Pd/C (5 wt.-%, nominally 50% water wet, 1.40 mmol of Pd, 0.02 eq.) were added to the solution. At 40° C., a solution of 22.5 g sodium acetate trihydrate (165 mmol, 2.59 eq.), 7.5 g of formic acid (80%, 130 mmol, 2.04 eq.) in 27.5 g of water was added over the course of 60 min. Then, the reaction mixture was stirred for 180 min at a constant temperature of 40° C.±2° C.

The Pd/C was filtered off via a suction filter (5-10 μm pore size). The obtained filtrate was concentrated via reduced-pressure evaporation. Then, the mixture was cooled and diluted with 600 ml of water. The pH value is set to 6-7 if necessary. Then, the reaction mixture was stirred overnight at RT. Afterwards, the reaction mixture was filtered and the obtained solid was washed twice with 30 ml water and dried at 40° C. in a vacuum drying oven. 12.29 g (75% HC, 12% HT) of a light red solid were obtained. The residual water content was determined to be 3.76%. Quantification was performed by means of HPLC.
Yield HC: 47.7%
Yield HT: 7.68%
HC:HT 6.21:1
HT:Pd 45.4:1

### Example 14

### 1,3-Butane Diol

20 g of hesperetin (96%, 63.5 mmol, 1 eq.) were dissolved in 300 g 1,3-butane diol and the solution was vigorously stirred under an inert gas atmosphere. Then, 5.98 g Pd/C (5 wt.-%, nominally 50% water wet, 1.40 mmol of Pd, 0.02 eq.) were added to the solution. At 40° C., a solution of 22.5 g sodium acetate trihydrate (165 mmol, 2.59 eq.), 7.5 g of formic acid (80%, 130 mmol, 2.04 eq.) in 27.5 g of water was added over the course of 60 min. Then, the reaction mixture was stirred for 180 min at a constant temperature of 40° C.±2° C.

The Pd/C was filtered off via a suction filter (5-10 μm pore size). The obtained filtrate was concentrated via reduced-pressure evaporation. Then, the mixture was cooled and diluted with 400 ml of water. The pH value is set to 6-7 if necessary. Then, the reaction mixture was stirred overnight at RT. Afterwards, the reaction mixture was filtered and the obtained solid was washed twice with 30 ml water and dried at 40° C. in a vacuum drying oven. 13.17 g (74.8% HC) of a light pink solid were obtained. The residual water content was determined to be 5.03%. Quantification was performed by means of HPLC.
Yield HC: 50.98%
Yield HT: —
HC:HT —
HT:Pd 45.4:1

### Example 15

### Taste Modulation in Different Bases

Based on an experimental design, 86 ice tea prototypes (drinkable preparations) were created that systematically varied regarding different sources of sweetness conveyed by sugars (e.g. HFCS (high fructose corn syrup), invert sugar syrup), sweeteners (Reb A, Reb D, Reb M, sucralose, acesulfame K) as well as hesperetin dihydrochalcone (HC) and hesperetin (HT) (the results are depicted in Table 2).

In order to investigate the effect of HT and HC on perceived sweetness dimensions, the 86 samples were profiled (sensory descriptive analysis) by a trained expert panel consisting of 12 panelists. The panelists were trained on an ice tea language with focus on sweetness attributes. Each attribute was scored on an unstructured line scale (10 cm) for its perceived intensity. To ensure high data reliability, every sample was tested twice. The sweetness attributes “sweetness onset”, “sweetness overall intensity” and “sweetness long-lastingness” were aggregated to a so-called sweetness factor using factor analysis (principal component analysis with VARIMAX rotation). The sweetness factor score runs from neg. infinity to positive infinity. By applying statistical modelling (JMP software: fit model: standard least squares, Analysis of Variance: DF 86, F 19.6907, p<0.0001; Rsquare 0.972) all significant main and two-way interaction effects were identified amongst which HC (p<0.0001), HT (p<0.0001) and the interaction of HC and HT (p<0.0001) were identified. These main and interaction effects were further tested in different bases (see table 2) using ANOVA, p<0.05; post hoc test: LSD. Table 2 shows that the interaction of HC and HT had the strongest positive effect on the sweetness factor followed by HC main effect and HT main effect coming third having the smallest positive effect (all sign. p<0.0001). Samples sharing the same letter are not significantly different.

TABLE 2
Results of the variance analysis of different samples.

| | | Hesperetin | | | |
|---|---|---|---|---|---|
| | Added sugar/ | dihydrochalcone | Hesperetin | | sign. |
| Sample | sweetener | (ppm) | (ppm) | ANOVA | differences |
| Sugar Free Ice | — | 0 | 0 | −3.7 | a |
| tea Base | | 10 | 0 | −1.3 | b |
| | | 0 | 3 | −3.6 | a |
| | | 10 | 3 | −0.9 | c |
| Invert Sugar | 2 wt. % Invert | 0 | 0 | −3.4 | a |
| Syrup Ice tea | sugar syrup | 10 | 0 | −1 | b |
| Base | | 0 | 3 | −3.3 | a |
| | | 10 | 3 | −0.5 | c |
| HFCS Ice tea | 2 wt. % High | 0 | 0 | −3.3 | a |
| Base | Fructose corn | 10 | 0 | −0.9 | b |
| | syrup | 0 | 3 | −3.2 | a |
| | | 10 | 3 | −0.4 | c |
| Reb A Ice tea | 60 ppm | 0 | 0 | −2.9 | a |
| Base | rebaudioside A | 10 | 0 | −0.5 | b |
| | | 0 | 3 | −2.8 | a |
| | | 10 | 3 | −0.1 | c |
| Reb A/Reb M | 60 ppm | 0 | 0 | −2.4 | a |
| Ice tea Base | rebaudioside A/ | 10 | 0 | 0 | b |
| | 45 ppm | 0 | 3 | −2.3 | a |
| | rebaudioside M | 10 | 3 | 0.5 | c |
| Reb A/Reb D | 60 ppm | 0 | 0 | −2.5 | a |
| Ice tea Base | rebaudioside A/ | 10 | 0 | −0.1 | b |
| | 60 ppm | 0 | 3 | −2.4 | a |
| | rebaudioside D | 10 | 3 | 0.4 | c |
| Reb M/Reb D | 45 ppm | 0 | 0 | −2.7 | a |
| Ice tea Base | rebaudioside M/ | 10 | 0 | −0.3 | b |
| | 60 ppm | 0 | 3 | −2.6 | a |
| | rebaudioside D | 10 | 3 | 0.1 | c |
| Sucralose Ice | 100 ppm | 0 | 0 | −3.2 | a |
| tea Base | sucralose | 10 | 0 | −0.8 | b |
| | | 0 | 3 | −3.1 | a |
| | | 10 | 3 | −0.3 | c |
| Acesulfame K | 60 ppm | 0 | 0 | −2.5 | a |
| Ice tea Base | | 10 | 0 | −0.1 | b |
| | | 0 | 3 | −2.4 | a |
| | | 10 | 3 | 0.4 | c |

### Example 16

### Influence of the Dosing of Hesperetin Dihydrochalcone and Hesperetin on Different Taste Aspects

In order to further validate the insights derived from Example 15, the interaction and main effects of hesperetin (HT) and hesperetin dihydrochalcone (HC) are to be shown in a setup in which a drinkable preparation with a tea base, 0.05 wt.-% lemon flavor, 4 wt.-% high fructose corn syrup, 0.1 wt.-% citric acid and 0.03 wt.-% trisodium citrate was prepared. That preparation was divided into four samples by varying the dosage of HC and HT resulting in one sample containing no HC and no HT (control), containing 10 ppm HC and no HT (HC main effect), containing no HC and 3 ppm HT (HT main effect) and, respectively, containing 10 ppm HC and 3 ppm HT (HT x HC interaction effect). The final preparations were profiled by an expert panel consisting of ten educated panelists (two measurements). Different sensorial attributes (ice tea language with focus on sweetness) were assessed and scored on an unstructured line scale (10 cm) for its perceived intensity. The results of the testing are shown below in Table 3.

TABLE 3
Sensory evaluation results

| | Lemon | | | Trisodium | | | | | | |
|---|---|---|---|---|---|---|---|---|---|---|
| Tea Base | Flavor | HFCS | Citric Acid | citrate | | | Onset | Overall | | |
| [wt.-%] | [wt.-%] | [wt.-%] | [wt.-%] | [wt.-%] | HT [ppm] | HC [ppm] | Sweetness | Sweetness | Syrupy | Acidity |
| 0.30 | 0.05 | 4 | 0.10 | 0.03 | 0 | 0 | 2.94 | 3.15 | 2.20 | 3.73 |
| 0.30 | 0.05 | 4 | 0.10 | 0.03 | 3 | 0 | 3.14 | 3.39 | 2.67 | 3.71 |
| 0.30 | 0.05 | 4 | 0.10 | 0.03 | 0 | 10 | 4.39 | 4.80 | 3.70 | 3.22 |
| 0.30 | 0.05 | 4 | 0.10 | 0.03 | 3 | 10 | 4.82 | 5.45 | 4.44 | 3.17 |

| | Lemon | | | Trisodium | | | | | | |
|---|---|---|---|---|---|---|---|---|---|---|
| Tea Base | Flavor | HFCS | Citric Acid | citrate | | | | | | |
| [wt.-%] | [wt.-%] | [wt.-%] | [wt.-%] | [wt.-%] | HT [ppm] | HC [ppm] | Fruity-Juicy | Tea | Bitter | Off-Notes |
| 0.30 | 0.05 | 4 | 0.10 | 0.03 | 0 | 0 | 3.40 | 4.23 | 2.94 | 1.96 |
| 0.30 | 0.05 | 4 | 0.10 | 0.03 | 3 | 0 | 3.60 | 4.30 | 2.93 | 1.85 |
| 0.30 | 0.05 | 4 | 0.10 | 0.03 | 0 | 10 | 3.88 | 4.08 | 2.58 | 2.25 |
| 0.30 | 0.05 | 4 | 0.10 | 0.03 | 3 | 10 | 4.25 | 3.76 | 2.25 | 2.53 |

| | Lemon | | | Trisodium | | | | | | | |
|---|---|---|---|---|---|---|---|---|---|---|---|
| Tea Base | Flavor | HFCS | Citric Acid | citrate | | | | | | | |
| [wt.-%] | [wt.-%] | [wt.-%] | [wt.-%] | [wt.-%] | HT [ppm] | HC [ppm] | | Balance | Complexity | Longlasting | Remarks |
| 0.30 | 0.05 | 4 | 0.10 | 0.03 | 0 ppm | 0 | ppm | 2.98 | 2.85 | 2.57 | |
| 0.30 | 0.05 | 4 | 0.10 | 0.03 | 3 ppm | 0 | ppm | 3.20 | 3.06 | 2.32 | |
| 0.30 | 0.05 | 4 | 0.10 | 0.03 | 0 ppm | 10 | ppm | 3.50 | 3.62 | 3.90 | lingering, |
| | | | | | | | | | | | sweetener |
| 0.30 | 0.05 | 4 | 0.10 | 0.03 | 3 ppm | 10 | ppm | 3.80 | 3.94 | 4.31 | pineapple |

### Example 17: Dosing Tests with an Excess of Hesperetin

A drinkable preparation with 7 wt.-% sucrose and 0.15 wt.-% citric acid was prepared. This preparation was used as a base composition for dosing tests with different dosing regimens of hesperetin dihydrochalcone (HC) and hesperetin (HT). The different samples were evaluated by an expert panel consisting of five educated panelists and ranked on a scale from 0 to 9. The results of the testing are shown below in Table 4. It can be derived from these data, that high dosing regimens of hesperetin in comparison to hesperetin dihydrochalcone exhibit strong off-tastes. At the same time, the sweetness impressions does not significantly increase, which results in an imbalanced taste profile.

TABLE 4
Sensory evaluation results of samples having hesperetin in excess

| Sample | | | | Description | | | | | | | |
|---|---|---|---|---|---|---|---|---|---|---|---|
| Sucrose | Citric Acid | HT | HC | Onset | Overall | | Long | Sweetener | | Off- | |
| [wt-%] | [wt-%] | [ppm] | [ppm] | Sweetness | Sweetness | Balance | Lasting | Like | Astringent | Notes | Comments |
| 7 | 0.15 | | | 4.5 | 5.5 | 5 | 0 | 0 | 1 | | |
| 7 | 0.15 | 3 | 10 | 5.5 | 7 | 4.5 | 1.5 | 1 | 1 | 1 | Slightly |
| | | | | | | | | | | | Vanilla, |
| | | | | | | | | | | | Phenolic |
| 7 | 0.15 | 20 | 10 | 6 | 7.5 | 4 | 2.5 | 1.5 | 2.5 | 1.5 | Slightly |
| | | | | | | | | | | | Vanilla, |
| | | | | | | | | | | | Phenolic |
| 7 | 0.15 | 30 | 10 | 6.5 | 8 | 3.5 | 4.5 | 2 | 3 | 3 | Vanilla, |
| | | | | | | | | | | | Phenolic |
| 7 | 0.15 | 40 | 10 | 6.5 | 7.5 | 3 | 5 | 2.5 | 3.5 | 4 | Phenolic, |
| | | | | | | | | | | | Smoky |
| 7 | 0.15 | 50 | 10 | 6.5 | 7.5 | 2.5 | 5 | 3 | 4 | 5 | Phenolic, |
| | | | | | | | | | | | Smoky |

1. A method for producing hesperetin dihydrochalcone comprising:
(i) providing hesperetin;
(ii) providing a catalyst selected from palladium catalysts, ruthenium catalysts, gold catalysts, platinum catalysts, copper catalysts, cobalt catalysts, iron catalysts, or combinations thereof;
(iii) providing a solvent comprising methanol, ethanol, an alkane diol having from 1 to 5 carbon atoms, or combinations thereof,
(iv) providing formic acid;
(v) reacting the formic acid of (iv) to form a formate;
(vi) mixing the hesperetin of (i), the catalyst of (ii), and the solvent of (iii) with the formate of (v) to obtain a reaction mixture;
(vii) heating the reaction mixture of (vi) to a temperature of 30 to 60° C. to convert hesperetin to hesperetin dihydrochalcone; and
(viii) removing the catalyst after heating the reaction mixture in (vii).

2. The method of claim 1, wherein the hesperetin provided in (i) and elemental metal of the catalyst of (ii) are in a molar ratio of 8:1 to 75:1 (hesperetin:elemental metal).

3. The method of claim 1, wherein the hesperetin provided in (i) and the formic acid provided in (iv) are in a molar ratio of 1:1 to 1:6 (hesperetin:formic acid).

4. The method of claim 1, wherein the hesperetin provided in (i) and the formate of (v) are in a molar ratio of 2:1 to 1:3.5 (hesperetin:formate).

5. The method of claim 1, wherein the hesperetin provided in (i) and the solvent provided in (iii) are in a molar ratio of 1:300 to 1:50 (hesperetin:solvent).

6. The method of claim 1, wherein the method further comprises:
(ix) removing solvent after heating the reaction mixture in (vii);
(x) adding water to the reaction mixture of (vii) to promote precipitation of hesperetin and/or hesperetin dihydrochalcone; and/or
(xi) purifying hesperetin and/or hesperetin dihydrochalcone after heating the reaction mixture in (vii).

7. The method of claim 1, wherein the method further comprises adding water to the reaction mixture and adjusting the pH so the reaction mixture with added water has a pH of 4 to 8, to promote precipitation of hesperetin and/or hesperetin dihydrochalcone.

8. The method of claim 7, wherein the method comprises purifying hesperetin and hesperetin dihydrochalcone after heating the reaction mixture in (vii) and drying a purified mixture of the hesperetin and hesperetin dihydrochalcone to a defined water content.

9. The method of claim 1, wherein the reaction mixture of (vii) is heated to a temperature of 35 to 50° C.

10. The method of claim 1, wherein the temperature of the reaction mixture in (vii) is maintained for 30 to 360 minutes.

11. The method of claim 1, wherein conversion of hesperetin to hesperetin dihydrochalcone (vii) is stopped before all of the hesperetin has been converted to hesperetin dihydrochalcone.

12. The method of claim 11, wherein the conversion is stopped such that the hesperetin and the hesperetin dihydrochalcone are in a weight ratio of 1:1 to 1:2000 (hesperetin:hesperetin dihydrochalcone).

13. The method of claim 11, further comprising determining the amount of hesperetin and the amount of hesperetin dihydrochalcone present after the conversion is stopped and adjusting the amounts to ensure the hesperetin and the hesperetin dihydrochalcone are in a weight ratio of 1:1 to 1:2000 (hesperetin: hesperetin dihydrochalcone).

14. A method for imparting or modifying a sweet taste impression to a substance or product comprising:
(a) providing the hesperetin dihydrochalcone obtained according to the method of claim 1; and
(b) mixing the hesperetin dihydrochalcone with the substance or product.

15. A method for imparting or modifying a sweet taste impression to a substance or product comprising:
(a) providing the hesperetin dihydrochalcone obtained according to the method of claim 6; and
(b) mixing the hesperetin dihydrochalcone with the substance or product.

16. The method of claim 1, wherein the catalyst is a Pd/C catalyst.

17. The method of claim 1, wherein the solvent comprises water and one or more of methanol, ethanol, a 1,2-alkane having from 1 to 5 carbon atoms, and a 1,3-alkane diol having from 1 to 5 carbon atoms.

18. The method of claim 1, wherein the solvent is a combination of water and ethanol.

19. The method of claim 1, wherein the formate is potassium formate, calcium formate, magnesium formate, ammonium formate, sodium formate, or combinations thereof.

20. The method of claim 1, wherein the formate is ammonium formate, sodium formate, or a combination thereof.
